(19) United States
(12) Patent Application Publication
(10) Pub. No.: US 2012/0035672 A1
(43) Pub. Date: Feb. 9, 2012

(54) CERAMIC CUTTING TEMPLATE

(76) Inventors: Roman Preuss, Kirchheim unter Teck (DE); Heinrich Wecker, Eckental (DE); Matthias Eschle, Wannweil (DE)

(21) Appl. No.: 13/260,923

(22) PCT Filed: Apr. 1, 2010
(86) PCT No.: PCT/EP10/54424

§ 371 Date: Oct. 11, 2011

(30) Foreign Application Priority Data
Apr. 1, 2009 (DE) 102009002084.5

(51) Int. Cl. A61B 17/56; C04B 35/10

(52) U.S. Cl. 606/86 R; 501/135

(57) ABSTRACT

A cutting template or a cutting block, preferably to a cutting template or a cutting block for use in medical technology.

Subject matter of the present invention is a cutting template or a cutting block, preferably a cutting template or a cutting block for use in medical technology.

During each knee-TEP-implantation, a so-called cutting template or cutting block is fixed on the femur. With this cutting template, normally, three cuts are carried out for adapting the femur surface to the geometry of the femur component. For each cut, there is one guide in the cutting template (3 or 4 cutting guides in 1 template). In this guide, the cut is carried out with an oscillating saw blade. Today, saw blades and cutting templates are principally made of biocompatible metal alloys.

Depending on the manufacturer, the guide rails in the cutting block have a width of 1.2-1.5 mm. Due to the oscillation of the saw blade and the friction occurring between saw blade and guide rail, a significant metal abrasion on the guide rail occurs. This metal debris can not be removed intraoperatively or only insufficiently from the wound. Hence, this debris can become the cause of infections and, in particular, can result in allergic reactions in the patient. For this reason it is important to principally reduce said debris and in particular if an implant reaction by the use of a ceramic femur component in a potential allergy sufferer is to be avoided.

According to the current state of knowledge, the majority of the metal debris is generated through wear on the guide rails in the cutting template. After a cutting template has been used approximately 20-40 times during knee-TEP-implantations, the guide rails show guide gaps which are increased by approximately 0.5-1.5 mm. As a result, the guide accuracy of the cutting template decreases significantly. The consequences for the surgeon correspond; a precise cut of the saw blade is no longer possible, alignment and evenness of the cut surfaces of the femur deviate increasingly. This results in larger gaps between the cut surfaces and the femur component. Said gaps have to be filled intraoperatively by a volume of bone cement that is larger than the usual volume which can have a negative effect on the durability of the system.

The object underlying the present invention is to eliminate the disadvantages of the cutting templates/cutting blocks of the prior art and, in particular:
to reduce the metal debris, wherein a reduction of the metal debris of up to 90% with respect to previous metal solutions is to be targeted;
to increase the service life of a cutting template and thus to save costs;
to reduce the risk of allergies and the risk of infections.

The object according to the invention was surprisingly achieved by a cutting template/a cutting block made of ceramics (hereinafter, the terms sinter-molded body or sintered body are also used for the cutting template according to the invention/cutting block according to the invention) with the features of the independent claims, Preferred configurations are to be found in the sub-claims. It was surprisingly found that the solution of the given object requires sinter-molded bodies with a very specific composition:
70 to 90 parts by volume of chromium-doped aluminum oxide (Al₂O₃:Cr),
12 to 22 parts by volume of Y-stabilized zirconium oxide (ZrO₂:Y) and
1 to 5 parts by volume of strontium aluminate of the formula SrAl₁₂₋ₓCrₓO₁₉ with variable Cr-doping.

With the teaching according to the invention, the metal debris is reduced by up to 90% compared to the previous cutting templates or cutting blocks made of metal. The service life of the cutting template or the cutting block according to the invention in use is considerably increased because only little wear on the cutting template occurs. This reduces the costs. Moreover, the allergy risk or the allergic reactions in patients and the risk of infections are reduced.

In one configuration of the invention, the constituents zirconium oxide and strontium aluminate are embedded in the aluminum oxide matrix.

Preferably, the strontium aluminate is present in the form of platelet-shaped crystallites and/or platelets.

In one inventive configuration, the material of the cutting template is additionally interspersed with whiskers and/or fibers or net-like structures or meshes from suitable materials.

The cutting template is preferably used in the field of medical technology, in particular during surgeries for treating a bone, in a preferred manner during a knee-TEP-implantation.

The advantages of the ceramic cutting template or of the ceramics from which it is made are:
The cutting template shows extremely low abrasive wear.
The material is biocompatible.
If the cutting template is labeled by a laser, the template is clearly visible and readable and therefore can reduce wrong handling during the use of the cutting template.
The cutting template has very good tribological properties.

It was surprisingly found that a cutting template with the following material composition is perfectly suited for the use in the field of medical technology.

| | | Parts by |
|---|---|---|
| Material composition | Formula | volume |
| Chromium-doped aluminum dioxide | Al₂O₃:Cr | 70%-90% |
| Y-stabilized zirconium oxide | ZrO₂:Y | 12%-22% |
| Strontium aluminate (with variable Cr- | SrAl₁₂₋ₓCrₓO₁₉ | 1%-5% |
| doping) | | |

The dominant micro-structural constituent of such a cutting template is the aluminum oxide. Thus, the property-determining features such as hardness, modulus of elasticity and thermal conductivity are close to the properties of pure aluminum oxide. The constituents zirconium oxide and strontium aluminate are embedded in the aluminum oxide matrix. The strontium aluminate forms characteristic platelet-shaped crystallites, platelets, which contribute significantly to increasing the strength.

The constituents zirconium oxide and strontium aluminate contribute to increasing the fracture toughness which is approximately 60% higher than in case of pure aluminum oxide. By these reinforcement components, the strength is increased by almost a factor of 2 and, at the same time, the damage tolerance increases, that is the cutting template's ability to maintain a high residual strength even in case of a potential damage.

During high mechanical load on the cutting template according to the invention, surprisingly, mechanisms are activated which inhibit or stop fracture propagation. The most important mechanism is the stress-induced conversion of the zirconium oxide from the tetragonal phase to the monoclinic phase. The volume increase of the zirconium oxide resulting from the conversion causes the formation of local compressive stress which counteracts the external tensile load and thus inhibits crack propagation.

The embedded platelets surprisingly deflect the crack path so that additional energy is absorbed during the crack propagation.

As special feature of the cutting template according to the invention is to be considered that the two mechanisms strengthen themselves mutually so that the effective increase of the fracture toughness is even higher as it would be expected by a simple addition of individual mechanisms.

Manufacturing of the cutting template is carried out by conventional ceramics technology.

The essential process steps are:
a) Preparing the powder mixture in water according to specified composition, use of liquefiers for preventing sedimentation.
b) Homogenizing in the dissolver (high-speed stirrer).
c) Grinding in an agitator ball mill, thereby increasing the specific surface of the powder mixture (=milling).
d) Adding organic binders.
e) Spray-drying, thereby generating free-flowing granules with defined properties.
f) Wetting the granules with water.
g) Pressing axially or isostatically.
h) Green machining, whereby in consideration of sinter shrinkage, the final contour is largely formed.
i) Prefiring, thereby shrinking to approx. 98% of the theoretical density. The still remaining residual pores are closed toward the outside.
j) Hot-isostatic pressing under high temperature and high gas pressure, thereby virtually complete final densification.
k) So-called clean burn; thereby, the imbalance of the oxygen ions in the ceramics generated during hot isostatic pressing is balanced.
l) Hard machining by grinding and polishing
m) Tempering.

The properties of the cutting template can still be enhanced by incorporations. It is possible to mix whiskers and/or fibers prior to demolding the cutting template into the material or to incorporate net-like structures or meshes into the material in the green state. The whiskers, fibers or net or meshes have to be made from a material which does not interact with the ceramic material in such a manner that a deterioration of the properties of the ceramic material occurs. Moreover, the material must not change during sintering in a manner that the material is damaged.

The cutting template surprisingly combines the in each case best properties of pure aluminum oxide and zirconium oxide: Hardness, ageing resistance, wetting behavior with respect to water and high thermal conductivity are properties which are known from sinter-molded bodies made of aluminum; high strength and fracture toughness, that is, damage tolerance are properties which are known from sinter-molded bodies made of zirconium oxide.

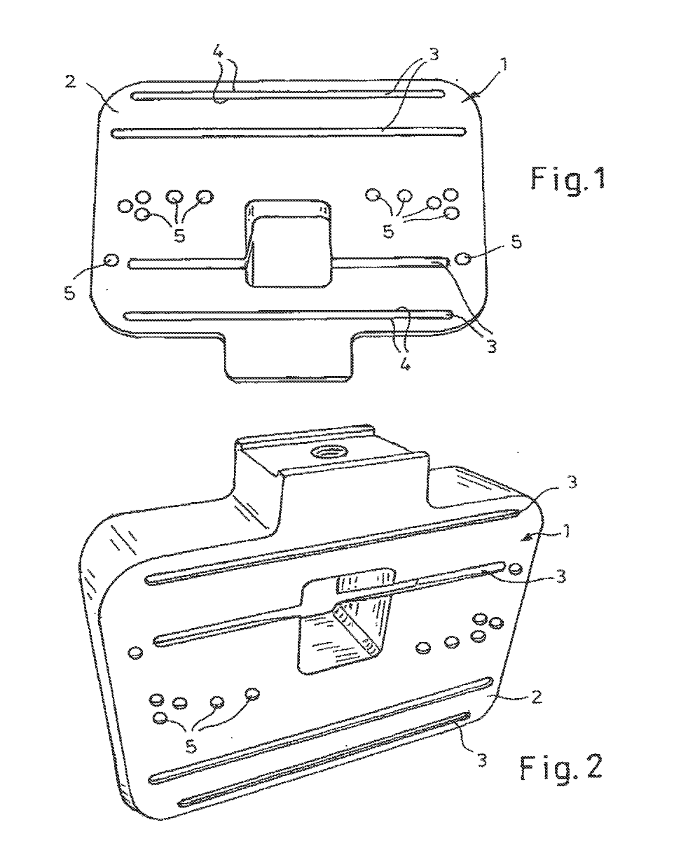

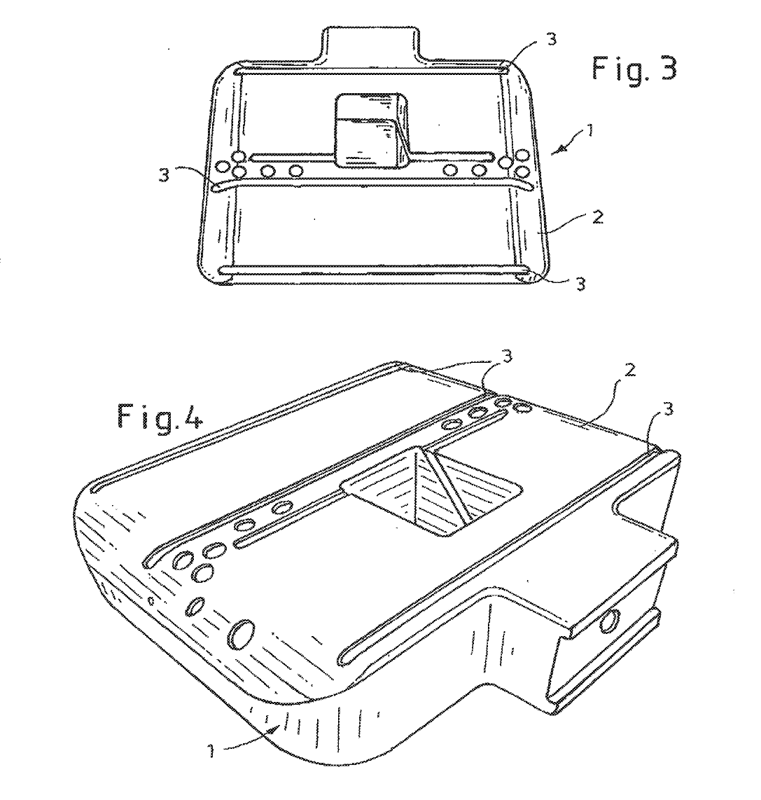

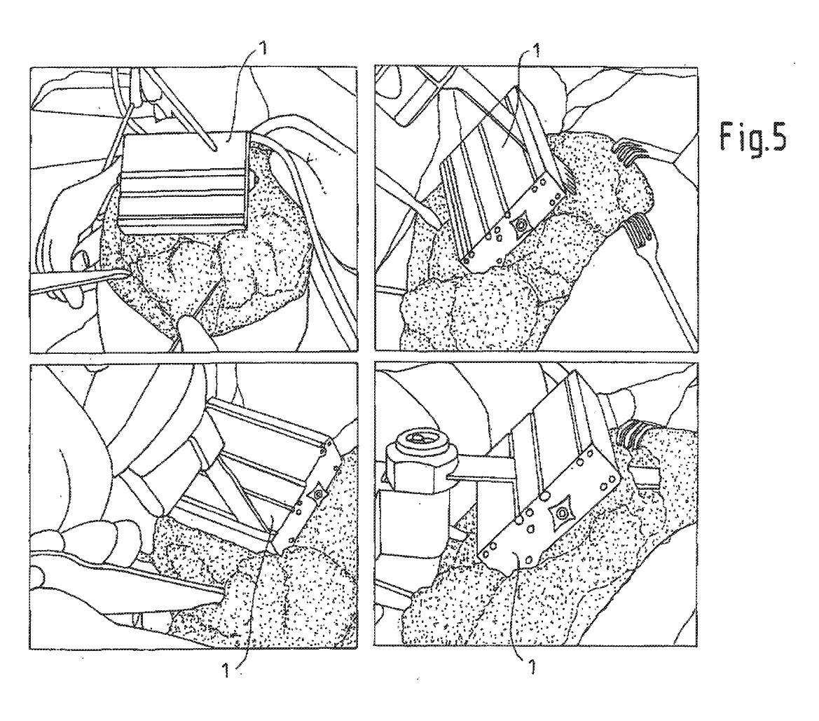

FIGS. 1 to 4 show a cutting template 1 according to the invention made of ceramics in different views FIG. 5 shows images with respect to the shape and the intraoperative use of a conventional cutting template made of metal.

FIGS. 1 to 4 show a cutting template 1 according to the invention which is also designated as cutting block. Such a cutting template serves for guiding a surgical saw blade during an implantation of an artificial knee joint.

The cutting template consists of a base body 2 which is provided with slot-like recesses 3 for inserting and precisely guiding a plate-shaped saw blade, wherein the slot-like recesses 3 have guide surfaces 4 which oppose each other. During the sawing process, the saw blade (see FIG. 5) rests against these guide surfaces 4. Through-holes 5 are drilled into the base body 2 which holes serve for screwing the cutting template 1 onto the femur.

Within the context of the present invention, the terms sinter-molded body/sintered body designate a ceramics in the form of a cutting template or cutting block or, respectively, a ceramics for the use as a cutting template or cutting block.

1-6. (canceled)

7. A cutting template, wherein said template contains 70 to 90 parts by volume of chromium-doped aluminum oxide of formula Al₂O₃:Cr, 12 to 22 parts by volume of Y-stabilized zirconium oxide of formula ZrO₂:Y and 1 to 5 parts by volume of strontium aluminate of formula SrAl₁₂₋ₓCrₓO₁₉ with variable Cr-doping.

8. The cutting template according to claim 7, wherein the zirconium oxide and the strontium aluminate are embedded in the aluminum oxide matrix.

9. The cutting template according to claim 7, wherein the strontium aluminate is present in at least one form selected from the group consisting of platelet-shaped crystallites and platelets.

10. The cutting template according to claim 7, wherein the material further comprises at least one member selected from the group consisting of whiskers, fibers, net-like structures and meshes from suitable materials.

11. A method comprising treating a bone with the cutting template according to claim 7.

12. A method comprising performing a knee-TEP-implantation with the cutting template according to claim 7.
